(19) United States
(12) Patent Application Publication
(10) Pub. No.: US 2008/0208201 A1
(43) Pub. Date: Aug. 28, 2008

(54) Apparatus for preparing a femur

(75) Inventors: Marie Moindreau, Caen (FR); Loic Pinot, Bayeux (FR); Richard Eddy Field, Surrey (GB); Neil Rushton, Cambridgeshire (GB)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Benoist Girard SAS, Herouville-saint-clair Cedex (FR)

(21) Appl. No.: 12/008,397

(22) Filed: Jan. 10, 2008

(30) Foreign Application Priority Data
Feb. 26, 2007 (GB) 0703691.6

(51) Int. Cl. A61B 17/58; A61B 17/56; A61F 5/00

(52) U.S. Cl. 606/96; 606/301; 606/87

(57) ABSTRACT

An apparatus for preparing a head portion of a proximal femur has a drill guide support having a cylindrical bore therein for engaging the head of a femur. The support has a central axis for coaxial alignment with a central axis of a neck of a proximal femur. A drill guide is mounted on a proximal end of the drill guide support, the drill guide having a first drill guide portion extending along an axis co-axial with the central axis of the base portion and a second drill portion extending along an axis forming an acute angle with the first drill portion central axis. A bone thickness indicator is mounted on the drill guide and is moveable towards and away from a neck of the femur along an axis forming an angle with the central axis of the drill guide support. A method of preparing the head of a femur which includes cutting a cylindrical surface around an axis the head of the femur, resecting a proximal portion of the femur in a plane perpendicular to the axis of the femoral head and mounting a drill guide support on the cylindrical surface of the femur is also disclosed.

### BACKGROUND OF THE INVENTION

This invention relates to apparatus for preparing a prosthetic stem cavity with inclined sides in the proximal end of a femur when performing a proximal epiphyseal femoral head replacement and resurfacing technique surgery.

In these techniques it is essential that the replacement surface for the head of the femur should be precisely located in both angular and translation positions of the axis of the femoral neck of the implant. To assist this, in some techniques, the surgeon inserts a pin in the lateral femur. The desired position of the pin will be known from pre-operative analysis of x-rays of the joint. The surgeon will measure the desired distance down the femur from the tip of the greater trochanter and the alignment pin is inserted through the vastus lateralis fibres. The alignment pin is inserted in a transverse direction into the mid-lateral cortex and directed upwardly towards the femoral head. The pin is left protruding so that an alignment guide can be hooked over the alignment pin.

The alignment guides of the kind described above generally comprise a hook or aperture which is placed over the alignment pin thus providing a good angular position for the axis of the implant in valgus, varus and ante-version of the neck. The guide will then be adjusted such that a annulated rod is located with the aperture therein directed down the mid-lateral axis of the femoral neck. A stylus having been set to the desired femoral component size is positioned such that it can be passed around the femoral neck. When the stylus can be passed around the femoral neck, the cannulated rod is locked in position. Once the guide is stabilized in this way fine adjustments can be made until the surgeon is happy that the guide is in the required position.

A guide wire can then be inserted through the cannulated rod. This guide wire is then used in the further surgery in which the femoral head is shaped to accept the prosthesis. This shaping involves removing the top of the head at an appropriate position and then machining the sides of the head using a sleeve cutter. These sleeve cutters are arranged such that the diameter cut will be correct for the replacement head size chosen and will bottom on the top of the cut head such that the teeth of the cutter do not dangerously over-sail the head-neck junction and cause soft tissue damage or neck notching.

Thus the machining procedure usually comprises the steps of drilling a well into the head of the femur, removing the drill, removing the top of the head of the femur, inserting a guide rod into the well, locating a sleeve cutter on the guide rod and cutting the head and optionally chamfer cutting the head. However, it will be understood that the order of the steps may be altered.

An alignment guide is generally used to ensure that the aperture drilled in the femoral head is both central to the femoral neck and at the correct angle of alignment thereto and that the shaping of the femoral head is accurate for the chosen head size. An alignment guide of this type is shown in U.S. Patent Publication No. 2007/0233136, the disclosure of which is incorporated herein by reference.

An example of this type of surgery is explained and shown in U.S. Pat. No. 6,524,343 the disclosure of which is incorporated herein by reference and includes resecting a femur at a position on the proximal side of its neck to locate a prosthetic femoral component which has a tapered insert portion and a proximal head portion. The insert portion is adapted for location in a prepared socket which effectively has inclined sides to provide a tapering opening.

The present invention provides apparatus for preparing such an opening.

### SUMMARY OF THE INVENTION

According to the present invention an apparatus or instrument for preparing a prosthetic stem cavity with inclined sides in the proximal end of a femur when performing proximal epiphyseal replacement technique surgery is provided. The instrument comprises a drill guide support which has a support part which has a first drill guide for drilling a lateral proximal/distal opening at an angle to the first opening adjacent the proximal end of the femur. The instrument has a lower part for location on and attachment to the prepared proximal end of a femur and a location device for locating the drill guide support at a predetermined angular position about a proximal distal axis on the femur.

The apparatus provides a method for accurately drilling the angulated bores in the femur which are subsequently shaped to provide the stem cavity.

If desired the drill guide support can also include a guide for transversely guiding a saw to remove proximal bone from the head of the femur before or after drilling. With this arrangement the upper part of the drill guide support means can include a saw slot.

In a preferred construction the lower part of the drill guide support is in the form of a distal attachment collar and the upper part of the drill guide support can be detachable from the lower part. The upper part of the drill guide support can be in the form of a spider element with radially projecting arms. Alternatively, the upper and lower parts of the drill guide support can be in the form of a monoblock construction. The drill guide support can also include an indicator for indicating the bone thickness between the predetermined distal end of the cavity and the outer surface of the femur.

The indicator for indicating the bone thickness is preferably detachably connected to the drill guide support and the indicator for indicating bone thickness may comprise a support member which carries adjustable distance indicator and in relation to which it can be adjusted to contact the outer surface of the bone. The adjustable distance indicator in a preferred construction comprises a sliding pin provided with distance indicating indicia.

Aspects of the present invention are provided by an apparatus for preparing a head portion of a proximal femur having a drill guide support. The support has a cylindrical bore therein with a central axis for coaxial alignment with a central axis of a neck of a proximal femur. A drill guide is mounted on a proximal end of the drill guide support. The drill guide has a first drill guide portion extending along an axis co-axial with the central axis of the base portion and a second drill portion extending along an axis forming an acute angle with the first drill portion central axis. A bone thickness indicator is mounted on the drill guide moveable towards and away from a neck of the femur along an axis forming an angle with the central axis of the drill guide support. The drill guide support has a saw blade guide slot extending in a direction perpendicular to the central axis of the base. The preferred bone thickness indicator is a sliding pin having distance marking indicia thereon. The drill guide support has at least three adjustable screws for engaging the femur and has a distally extending pointer alignable with a calcar of the femur. The bone thickness indicator may be releasably mounted to the drill guide support so that it may be moved in a proximal distal direction. A method of preparing the head of a femur is also provided and involves cutting a cylindrical surface around an axis the head of the femur, resecting a proximal portion of the femur in a plane perpendicular to the axis of the femoral head, mounting a drill guide support on the cylindrical surface of the femur, drilling at least one cavity on the head of the femur using the drill guide and sliding a pin mounted on the drill guide to indicate the bone thickness between the distal end of the cavity and the outer surface of the femur. The method may also include drilling two cavities in the head of the femur, the cavities intersecting at an angle. The drill guides for drilling the cavity are located on the drill guide support. The pin is provided with distance marking indicia. The preferred mounting of the drill guide to the cylindrical femur surface is accomplished with three spaced adjustable screws mounted on the drill guide which screws engage the cylindrical surface. The drill guide support is located circumferentially about the femur by aligning an indicia on the drill guide support with the calcar of the femur. The proximal portion of the femur may be resected with a saw guide surface located on the drill guide support.

As used herein when referring to bones or other parts of the body, the term “proximal” means close to the heart and the term “distal” means more distant from the heart. The term “inferior” means toward the feet and the term “superior” means toward the head. The term “anterior” means toward the front part or the face and the term “posterior” means toward the back of the body. The term “medial” means toward the midline of the body and the term “lateral” means away from the midline of the body.

### BRIEF DESCRIPTION OF THE DRAWINGS

The invention can be performed in various ways and two embodiments will now be described by way of example and with reference to the accompanying drawings in which:

FIG. 1 is a diagrammatic representation of the proximal end of a femur showing how it is prepared to accept the apparatus according to the invention;

FIG. 2 is an exploded isometric view from above showing the various parts of the apparatus according to the invention;

FIG. 3 is a view similar to FIG. 2 with the parts assembled;

FIG. 4 is an isometric view from above of an alternative construction;

FIG. 5 is an exploded isometric view of the construction shown in FIG. 4.

FIG. 6 is a side elevation of the construction shown in FIG. 4

FIG. 7 is an exploded side view of the parts as shown in FIG. 5;

FIG. 8 is a diagrammatic side view showing how some of the parts are assembled onto the femur;

FIG. 9 shows all the parts of the construction shown in FIGS. 4 to 7 assembled on the femur; and

FIG. 10 is a diagrammatic cross-sectional representation of a prosthetic component for use in a prosthetic stem cavity prepared by apparatus according to the invention.

### DETAILED DESCRIPTION

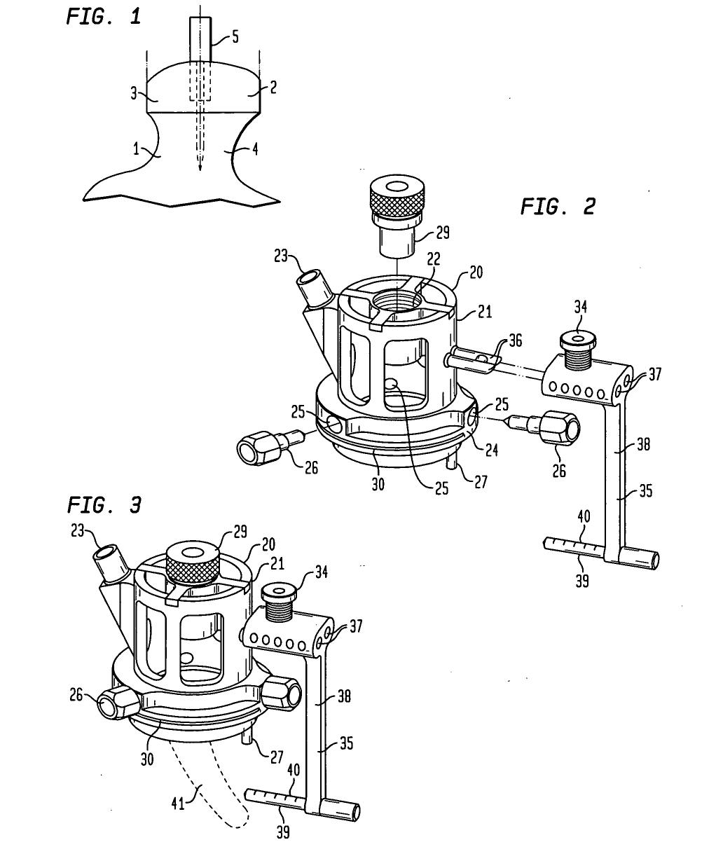

In order to carry out proximal epiphyseal femoral head replacement technique surgery on the end of a femur indicated by reference numeral 1 in FIG. 1 the femoral head, indicated by reference numeral 2, is first prepared by machining it with a cylindrical cutter (not shown) mounted on a guide pin to provide a cylindrical portion 3. To accurately locate the cylindrical cutter a proximal/distal opening is drilled in the head to receive a guide wire and this opening is subsequently enlarged to receive a guide pin 5 on which the cylindrical cutter is located. The neck of the femur is indicated by reference numeral 4.

FIG. 10 shows diagrammatically how a typical femoral head component which has a tapered insert portion 11 and a head 12 is fitted into a cavity 13 in the proximal end of the femur 1 (indicated by broken lines in FIG. 10) according to this type of surgery.

In order to provide the cavity 13 a hole is drilled into the prepared femur along an axis 14 which is substantially co-axial with the proximal/distal axis of the neck end of the femur. A second opening is also drilled along the line 15 which is at an angle to the line 14 to provide the basis for the tapering socket and corresponds to the angle of the distal stem of the implant.

As shown in FIGS. 2 and 3 a first embodiment of the invention is of a monoblock construction and comprises drill guide support 20 which has an upper portion 21 in the form of a cylindrical barrel and which has a first drill guide 22 for drilling a proximal/distal opening in the proximal end of the femur along the line 14 as shown in FIG. 10. The upper part 21 also has a second drill guide 23 for drilling a lateral/proximal distal opening at an angle to the first opening adjacent the proximal end of the bone and which would produce the opening along the centerline as shown in FIG. 10.

A lower part of the drill guide support 20 is indicated by reference numeral 24 and is the form of a distal attachment collar which is dimensioned to fit accurately over the machined cylindrical portion 3 of the prepared head 2. Three tapped bores 25 are provided into which attachment screws 26 can be threaded to engage the cylindrical bone portion 3 and locate and attach the drill guide support to the prepared head 2 of the femur.

A locator for locating the drill guide support 20 at a predetermined circumferential angular position about a proximal distal axis on the femur, for example the line 14 of FIG. 10 is provided by a downwardly projecting pointer 27 which can be aligned with the calcar, indicated in FIG. 10 by reference numeral 28.

The drill guide 22 is provided with a removable centering bushing 29 which can be located in drill guide 22 and which is dimensioned to fit over guide pin 5 to align the drill guide support 20 when being fitted. It is then removed along with the guide pin 5.

In order to trim the head 2 a transversely extending saw slot 30 is provided in the lower part 24 and this can be utilized to remove bone from the head of the femur before or after drilling.

The drill guide support 20 also includes an indicator for indicating the bone thickness between the predetermined distal end of the cavity, indicated by reference numeral 31 of FIG. 10, and the outer surface of the bone of the femur, indicated by reference numeral 32. The bone thickness indicator reference numeral 35 is detachably connected to drill guide support 20 by two projecting location pins 36 which can engage co-operating openings 37 in a support member 38. The pins are held in position by a spring loaded locking pin 34. The lower end of the support carries an adjustable distance indicator in the form of a sliding pin 39 and which can be adjusted to contact the outer surface of the bone 32 (see FIG. 10). Sliding pin 39 is provided with distance indicating indicia 40.

In FIG. 3 support member 38 is shown in place on the drill guide support 20. With the apparatus in place on the prepared femur head and knowing the dimensions of the various parts it is possible to move sliding pin 39 until it engages the side of the bone opposite the pre-estimated position of the end of the drilled opening 13. The distance indicating indicia 40 will now provide a reading which will give the thickness of the bone between the end of the opening and the bone wall. Thus, this bone thickness indicator is used to confirm the bone thickness before drilling takes place.

When the openings have been drilled the apparatus is removed and a rasp one size smaller than the definitive size of the implant stem can be used to enlarge the cavity taking care not to enlarge the cavity beyond the medial and lateral limits of the original holes. Such a rasp is shown by broken line 41 in FIG. 3.

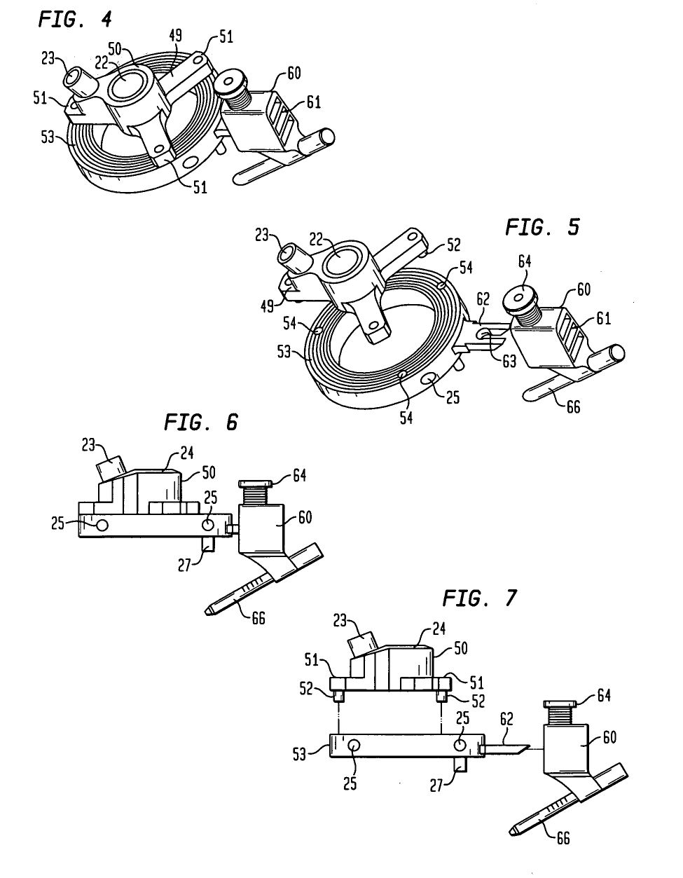

FIGS. 4 to 9 show an alternative construction embodying the invention and the same reference numerals are used to define similar parts as shown in FIGS. 2 and 3.

In this construction the drill guides 23 and 22 are carried by drill guide support 50 in the form of a spider portion 49 with radially projecting arms 51. Each of the arms has a location pin 52 (best seen in FIG. 7) which can engage in the upper surface of a lower collar part 53 which is in the form of a detachable distal attachment collar. The pins 52 can engage in openings 54 in the attachment collar 53 to locate the spider 49 in position thereon and tapped openings 25 are again provided to receive bolts 26 (not shown in FIGS. 4 to 9) to locate the distal attachment collar in place on the prepared femur head.

With this embodiment, the indicator for indicating the bone thickness comprises a support member 60 provided with a series of slots 61 which can appropriately engage a projecting lug 62 on the side of collar 53 to effectively adjust the proximal/distal length of support member 60. The lug has an opening 63 to receive a spring loaded locking pin 64 to hold support member 60 in place thereon. The sliding pin 66 in this construction is arranged at an angle to provide an appropriate engagement with the outer surface of the bone.

The location pin 27 is again provided on the lower part of the support 50 but no saw slot is included. The upper surface of the attachment collar 53, however, acts as a guide for a saw for removing proximal bone from the head of the femur when required. In certain circumstances the means for indicating the bone thickness between the predetermined distal end of the cavity and the surface may not be necessary and thus the projecting lug 62 on the side of collar 53 can be omitted.

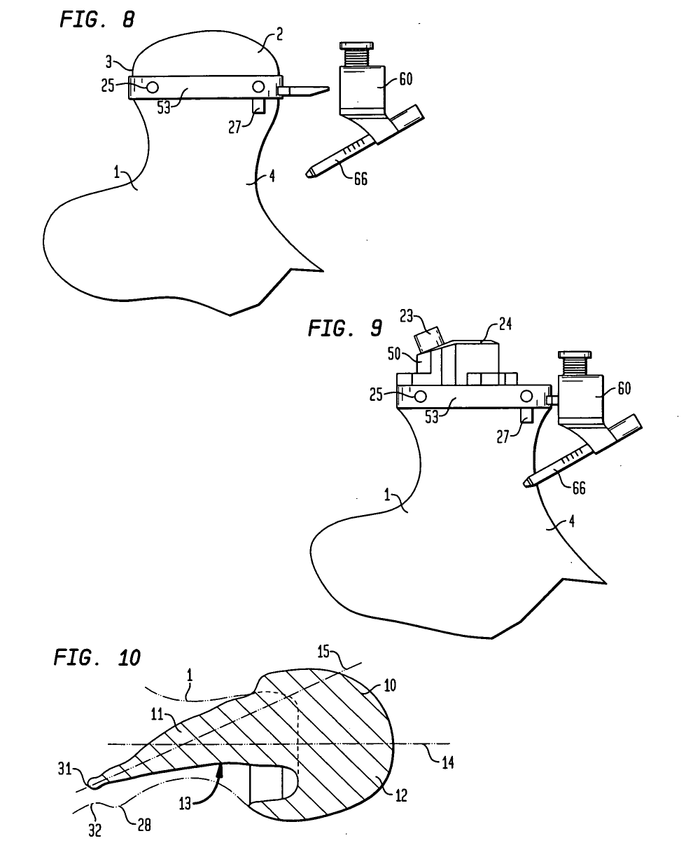

FIG. 8 and 9 show how this embodiment is used The head 2 of the femur is prepared as shown in FIG. 1 with a cylindrical portion 3 and the distal attachment collar 53 is placed in position using the screws 26. With the collar located in the appropriate position using the guide pin 27 the screws 26 are inserted and the upper surface of the collar is used as a guide to remove proximal bone from the head of the femur. The spider portion 49 is now placed in position as is the bone thickness indicator 60, the positions checked and drilling can then take place in a similar manner to that described with regard to FIGS. 2 and 3.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. An apparatus for preparing a head portion of a proximal femur comprising:
a drill guide support having a cylindrical bore therein having a central axis for coaxial alignment with a central axis of a neck of a proximal femur;
a drill guide mounted on a proximal end of the drill guide support, the drill guide having a first drill guide portion extending along an axis co-axial with the central axis of the drill guide support and a second drill portion extending along an axis forming an acute angle with the first drill guide portion axis; and
a bone thickness indicator mounted on the drill guide support moveable towards and away from a neck of the femur along an axis forming an angle with the central axis of the drill guide support.

2. The apparatus as set forth in claim 1 wherein the drill guide support has a saw blade guide slot extending in a direction perpendicular to the central axis of the drill guide support.

3. The apparatus as set forth in claim 1 wherein the bone thickness indicator is a sliding pin having distance marking indicia thereon.

4. The apparatus as set forth in claim 1 wherein the drill guide support has at least three screws for engaging the femur.

5. The apparatus as set forth in claim 4 wherein the drill guide support has a distally extending pointer alignable with a calcar of the femur.

6. The apparatus as set forth in claim 1 wherein the apparatus comprises means for releasably mounting the bone thickness indicator to the drill guide support.

7. A method of preparing the head of a femur comprising:
cutting an outer cylindrical surface around an axis the head of the femur;
resecting a proximal portion of the femur in a plane perpendicular to the axis of the femoral head;
mounting a drill guide support on the cylindrical surface of the femur;
drilling at least one cavity in the head of the femur using a drill guide mounted on the drill guide support; and
sliding a pin mounted on the drill guide support to indicate the bone thickness between the distal end of the cavity and the outer surface of the femur.

8. The method as set forth in claim 7 further comprising drilling two cavities in the head of the femur, the cavities intersecting at an angle.

9. The method as set forth in claim 7 wherein the pin is provided with distance marking indicia.

10. The method as set forth in claim 7 wherein the mounting of the drill guide support to the cylindrical femur surface is accomplished with three spaced screws mounted on the drill guide support which screws engage the cylindrical surface.

11. The method as set forth in claim 7 further comprising locating the drill guide support circumferentially about the femur by aligning an indicia on the drill guide support with the calcar of the femur.

12. The method as set forth in claim 7 further comprising resecting the proximal portion of the femur with a saw guide surface on the drill guide support.

13. The method as set forth in claim 7 further comprising locating two drill guides on the drill guide support.

14. The method as set forth in claim 13 further comprising drilling two cavities in the head of the femur, the cavities intersecting at an angle.

15. An apparatus for forming an asymmetrical cavity in the proximal femur comprising
a drill guide support for engaging an outer surface of the femoral head;
first and second drill guide mounted on the drill guide support each extending along a respective first and second axis, the first and second axis forming an acute angle therebetween; and
a bone thickness indicator mounted on the drill guide support movable into contact with the proximal femur adjacent a medial most part of the cavity formed by the insertion of the first and second drill guide into the proximal femur.

16. The apparatus as set forth in claim 15 wherein the drill guide support has a saw blade guide slot extending in a direction perpendicular to the central axis of the drill guide support.

17. The apparatus as set forth in claim 15 wherein the bone thickness indicator is a sliding pin having distance marking indicia thereon.

18. The apparatus as set forth in claim 15 wherein the drill guide support has at least three screws for engaging the femur.

19. The apparatus as set forth in claim 18 wherein the drill guide support has a distally extending pointer alignable with a calcar of the femur.

20. The apparatus as set forth in claim 15 wherein the apparatus comprises means for releasably mounting the bone thickness indicator to the drill guide support.

21. An apparatus for preparing a prosthetic stem cavity with inclined sides in the proximal end of a femur when performing proximal epiphyseal replacement technique surgery comprising drill guide support means which has a support part which has a first drill guide for drilling a proximal/distal opening in the proximal end of the bone and a second drill guide for drilling a lateral proximal/distal opening at an angle to said first opening adjacent the proximal end of the bone, a lower part for location on and attachment to the prepared proximal end of a femur and means for locating said drill guide support means at a predetermined angular position about a proximal distal axis on the femur.

22. The apparatus as claimed in claim 21 in which the drill guide support means include means for transversely guiding a saw to remove proximal bone from the head of the femur before or after drilling.

23. The apparatus as claimed in claim 22 in which the said upper part includes a saw slot.

24. The apparatus as claimed in claim 21 in which the lower part of the drill guide support means is in the form of a distal attachment collar.

25. The apparatus as claimed in claim 21 in which said upper part of the drill guide support means is detachable from the said lower part.

26. The apparatus as claimed in claim 25 in which said upper part of the drill guide support means is in the form of a spider with radially projecting arms.

27. The apparatus as claimed in claim 21 in which the upper and lower parts of the drill guide support means are in the form of a monoblock construction.

28. The apparatus as claimed in claim 21 in which said drill guide support means includes means for indicating the bone thickness between the predetermined distal end of the cavity and the outer surface of the femur.

29. The apparatus as claimed in claim 28 in which the means for indicating bone thickness is preferably detachably connected to said drill guide support means.

30. The apparatus as claimed in claim 28 in which the means for indicating bone thickness comprises a support member which carries adjustable distance indicating means and in relation to which it can be adjusted to contact the outer surface of the bone.

31. The apparatus as claimed in claim 30 in which the adjustable distance indicating means comprises a sliding pin provided with distance indicating indicea.
